(19) United States
(12) Patent Application Publication
(10) Pub. No.: US 2023/0302213 A1
(43) Pub. Date: Sep. 28, 2023

(54) Method for Priming an Extracorporeal Blood Circuit and Devices

(71) Applicant: Fresenius Medical Care Deutschland GmbH, Bad Homburg (DE)

(72) Inventor: Bernard Canaud, Montpellier (FR)

(21) Appl. No.: 18/024,239

(22) PCT Filed: Aug. 30, 2021
(86) PCT No.: PCT/EP2021/073845

§ 371 Date: Mar. 1, 2023

(30) Foreign Application Priority Data
Sep. 2, 2020 (EP) 20194067.3

(51) Int. Cl. A61M 1/36

(52) U.S. Cl. CPC A61M 1/3649; A61M 1/3661; A61M 2205/3368

(57) ABSTRACT

The present disclosure relates to a method for priming an extracorporeal blood circuit using a blood treatment apparatus comprising a dialysis liquid preparation system having, a source of water, a source of bicarbonate concentrate and a source of acid concentrate including sodium chloride while not having a source of sodium chloride only, the method including preparing a priming solution from said source (of water and said source of acid only to obtain an acid/water solution, wherein bicarbonate from the source of bicarbonate concentrate is absent from said solution, connecting an arterial line section of the extracorporeal blood circuit to an outlet of the dialysis liquid preparation system of the blood treatment apparatus, and filling the extracorporeal blood circuit with said priming solution.

### CROSS-REFERENCE TO RELATED APPLICATIONS

The present application is the national stage entry of International Patent Application No. PCT/EP2021/073845, filed on Aug. 30, 2021, and claims priority to Application No. EP 20194067.3, filed in the European Patent Office on Sep. 2, 2020, the disclosures of which are expressly incorporated herein in their entirety by reference thereto.

### TECHNICAL FIELD

The present disclosure relates to a method as described herein. It also relates to a control device or closed-loop control device as described herein and a blood treatment apparatus as described herein. Furthermore the present disclosure relates to a digital storage medium, a computer program product, and a computer program.

### BACKGROUND

Extracorporeal blood treatment using dialysis is known from practice. Whereby the patient’s blood is withdrawn and extracorporeally fed along a blood circuit and through, e.g., a blood filter. The blood filter comprises a blood chamber through which blood is guided, and a dialysis liquid chamber, through which dialysis liquid is guided. Both chambers are separated from each other by a semi-permeable membrane. Blood and dialysis liquid are mostly guided through the blood filter by the counter-current principle. The blood is purified in the blood filter, on exiting the blood filter the dialysis liquid, from now on referred to as effluate or effluent, is regarded as used and is discarded. In addition to the dialysate, the effluent to be discarded also comprises filtrate (or ultra-filtrate), which comprises water that has been withdrawn from the blood in the blood filter. Filtrate and dialysate will be referred to individually or collectively in the following simply as effluent. In addition to acute cases, dialysis is mainly used with patients who have end-stage renal failure.

Typically before a dialysis patient is connected to the dialysis equipment the extracorporeal circuit has to be primed. The purpose of priming the circuit is to remove air from the blood lines and the dialyzer as well as to remove possible fragments of remaining sterilising agents or other residuals from the disposables elements, such as blood lines and dialyzers that form the extracorporeal circuit, before the patient is connected.

### SUMMARY

In some aspects, the present disclosure provides a method for priming an extracorporeal blood circuit before the blood treatment session starts.

Furthermore, a control device or closed-loop control device, a blood treatment apparatus, a suitable digital storage medium, a suitable computer program product and a suitable computer program are described.

An exemplary method described herein relates to priming an extracorporeal blood circuit using or providing a blood treatment apparatus comprising a dialysis liquid preparation system. The dialysis liquid preparation system comprises or consists of a source of water, a source of bicarbonate concentrate (e.g., one container, bag or the like) and a source of acid concentrate (e.g., one container, bag or the like), the latter including sodium chloride, an acid, for example citric or acetic acid and further electrolytes, like magnesium chloride and or calcium chloride. The dialysis liquid preparation system does not use a source of sodium chloride only. The dialysis liquid preparation system is configured to prepare a dialysis liquid from those three sources mentioned (water, said bicarbonate concentrate source and said acid concentrate source) only.

The extracorporeal blood circuit comprises an arterial line section, connectable to a patient. It serves to withdraw blood from the patient. Further, the extracorporeal blood circuit comprises a venous line section which is connectable to the patient for returning the blood to her. Moreover, a blood side compartment or blood chamber of a blood filter is comprised by the extracorporeal blood circuit.

The method can encompass a first phase as follows:
a) preparing a priming solution from said source of water and said source of acid concentrate only to obtain an acid/water solution, wherein bicarbonate from the source of bicarbonate concentrate is absent from said solution;
b) connecting the arterial line section to an outlet of the dialysis liquid preparation system of the blood treatment apparatus, e.g., to the substitution port or another port of the blood treatment apparatus; and
c) filling the extracorporeal blood circuit with said priming solution.

The present disclosure further relates to a control device or closed-loop control device. The device is configured or programmed to carry out and/or to initiate the method described herein, in particular in each of the embodiments described herein and in each possible combination of the herein disclosed features, in particular method steps.

The control device or closed-loop control device may comprise devices or may be connected in signal communication with such devices, which can execute the individual method steps or method features which are disclosed herein - and particularly in the claims - and are designed, configured and/or programmed accordingly for this purpose.

The present disclosure further relates to a blood treatment apparatus for the extracorporeal treatment of a patient’s blood which comprises a control device or closed-loop control device as described herein.

A digital, in particular non-volatile, storage medium, particularly in the form of a machine readable carrier, particularly in the form of a diskette, storage card, CD, DVD Blu-ray disc or (E)EPROM, FRAM (Ferroelectric RAM) or SSD (Solid-State-Drive) with electronically readable control signals, may be configured to interact with a programmable control device or closed-loop control device in such a way that the, particularly mechanical, steps of the method described herein are initiated or executed.

Thereby, all, any or several of the steps, in particular mechanical steps, of the method described herein may be initiated or executed.

Alternatively or additionally, the digital storage medium described herein may be configured so that a conventional control device or closed-loop control device can be reprogrammed into a control device or closed-loop control device described herein.

A computer program product as described herein comprises a transient, volatile program code or a program code saved on a machine readable carrier for initiating or executing the, particularly mechanical, steps of the method described herein, when the computer program product is running on a control device or closed-loop control device.

The computer program product described herein can be understood, for example, as a computer program stored on a carrier, an embedded system being a comprehensive system with a computer program (e.g., electronic device with a computer program), a network of computer-implemented computer programs (e.g. client/server-system, a cloud computing system etc.), or a computer on which a computer program is loaded, runs, stored, executed or developed.

Alternatively or additionally hereto, the computer program product described herein may be configured to reprogram a conventional control device or closed-loop control device into a control device or closed-loop control device described herein.

The term “machine-readable carrier” as it is used herein, refers in certain embodiments to a carrier, which contains data or information interpretable by software and/or hardware. The carrier may be a data carrier, such as a diskette, a CD, DVD, a USB stick, a flashcard, an SD card or the like, as well as any other storage referred to herein or any other storage medium referred to herein.

A computer program described herein comprises a program code to initiate or execute the steps of the method described herein, particularly the mechanical steps, when the computer program is running on a control device or closed-loop control device. A computer program can be understood to mean, for example, a physical, ready-for-distribution software product that comprises a program.

Alternatively or additionally, the computer program described herein may be configured in order to reprogram a conventional control device or closed-loop control device into a control device or closed-loop control device as described herein.

It also applies to the computer program product described herein and the computer program described herein, that all, any, or several of the steps, in particular mechanical steps, of the method described herein may be initiated or executed.

Embodiments may comprise one or several of the aforementioned or following features in any combination, unless the person skilled in the art recognizes their combination as technically impossible.

In all of the following statements, the use of the expression “may be” or “may have” and so on, is to be understood synonymously with “preferably is” or “preferably has,” and so on respectively, and is intended to illustrate an embodiment according to the present disclosure.

Whenever numerical words are mentioned herein, the person skilled in the art shall recognize or understand them as indications of a numerical lower limit. Unless it leads the person skilled in the art to an evident contradiction, the person skilled in the art shall comprehend the specification for example of “one” as encompassing “at least one”. This understanding is also equally encompassed by the present disclosure as the interpretation that a numeric word, for example, “one” may alternatively mean “exactly one”, wherever this is evidently technically possible for the person skilled in the art. Both are encompassed by the present disclosure and apply herein to all numerical words used.

When it is disclosed herein that the subject-matter according to the present disclosure comprises one or several features in a certain embodiment, it is also respectively disclosed herein that the subject-matter according to the present disclosure does, in other embodiments, likewise according to the present disclosure, explicitly not comprise this or these features, for example, in the sense of a disclaimer. Therefore, for every embodiment mentioned herein it applies that the converse embodiment, e.g., formulated as negation, is also disclosed.

Whenever an embodiment is mentioned herein, it is then an exemplary embodiment according to the present disclosure.

In some embodiments, the temperature of the priming solution is kept below 35° C., preferably between 25° C. and 30° C. during the first phase of the method described herein.

In several embodiments, the method described herein further encompasses adding bicarbonate from the source of bicarbonate concentrate to the priming solution in a second phase following the first phase.

In some embodiments of the method, the temperature of the priming solution is raised to more than 30° C., preferably more than 35° C., for example to 36° to 37°, during the second phase.

In several embodiments of the method, the priming solution prepared in the first phase and/or prepared in the second phase is being circulated within the extracorporeal circuit for at least 5 minutes before the priming is considered as completed.

In some embodiments, the blood treatment apparatus described herein is embodied as a hemodialysis apparatus, hemofiltration apparatus or hemodiafiltration apparatus. In particular, it is embodied as an apparatus for acute or chronic renal replacement therapy.

In several embodiments, the blood treatment apparatus comprises sensors, arranged and configured in order to measure the sodium concentration of the priming solution.

In some embodiments, the blood treatment apparatus described herein comprises sensors inserted upstream and/or downstream of the dialyzer of the blood treatment apparatus to measure the electrolyte and/or fluid balance, e.g., on the dialysis liquid side or machine side and/or on the blood side. They can be used to determine the sodium concentration as discussed herein.

In some embodiments, the control device or closed-loop control device described herein may be programmed and/or configured in order to control or regulate the blood treatment device in addition to carrying out or causing or initiating the method described herein in cooperation with other devices.

In some embodiments, the sources or containers are not seated in a removable carrier, vehicle, support or caddy.

In several embodiments the blood treatment apparatus does not comprise a source or container detachably removable from the dialysis system which is configured to hold one or more other containers, optionally including one or more connectors for fluid connection from the containers to the dialysis system and which may in particular denoted as a “caddy”.

In some embodiments, the sources are no detachable containers but, rather, bags or the like.

Whenever a suitability or a method step is mentioned herein, the present disclosure also comprises corresponding programming or a configuration of a suitable device according to the present disclosure or a section thereof.

Some or all of the embodiments of the present disclosure may have one, several or all of the advantages listed above and/or below.

By using of the method described herein, air is removed from the blood lines and the blood chamber of the dialyzer of the extracorporeal blood circuit. Also, possible fragments of remaining sterilising agents or other residuals are removed from the disposable elements, such as blood lines and dialyzers that form the extracorporeal circuit, before the patient is connected.

In the past, saline solutions from bags of physiological saline solution were used to prime the extracorporeal blood circuit. Later, dialysis equipment was available that could prepare the substitution fluid for hemofiltration or hemodiafiltration on-line while obtaining a sterile and pyrogen-free fluid. Such on-line prepared substitution fluid has been also used for priming, which also is cost saving and convenient from a handling point of view.

However, due to drawbacks caused by using substitution fluid for priming, after which the patients often experienced problems, e.g., not feeling well, clinics have often gone back to priming with saline from bags after having experienced such problems.

For using remarkably less bicarbonate with the priming solution prepared described herein, a priming solution could be suggested by the present disclosure.

Advantageously, an almost bubble-free rinsing solution, which applies to both bicarbonate and CO₂ bubbles, may be provided and used before starting a dialysis treatment. Hence formation of microbubbles that could remain in the extracorporeal blood circuit even beyond the priming procedure is minimised or avoided. As a result, it is less likely that microbubbles will be present in the extracorporeal blood circuit once the priming process has been completed. This may contribute to the patient’s safety and may help to reduce nursing preparation time. In particular, typical nursing steps such as setting up the tubing, dialyzer, or the blood treatment apparatus altogether, and degassing of the same may be facilitated, and time needed for such steps may be significantly reduced.

A further advantage of the present disclosure may be seen in the two-step use of the on-line produced bicarbonate dialysis liquid. It may be firstly used as an acidic liquid for priming and maintaining the extracorporeal blood circuit and secondly as a regular bicarbonate dialysis liquid during a dialysis session.

As an acid solution is used for priming the extracorporeal blood circuit, an additional interpatient disinfection thereof may be omitted. This may help to save time and money.

Following the steps of the method described herein, less bicarbonate may be needed which is of benefit both for costs and logistics.

When using the methods, systems, and devices disclosed herein, calcium/magnesium carbonate precipitation may advantageously be prevented. A calcification of the hydraulic circuit may, therefore, be advantageously avoided.

Also, the risk of micro-bacterial or micro-organism contamination and/or bacterial growth within the extracorporeal circuit may be advantageously prevented. This may contribute to the patient’s safety.

Providing and monitoring the conductivity cell as disclosed herein may further contribute to the patient’s safety.

A further advantage of the present disclosure is that it is easy to implement.

All advantages achievable with the method described herein can also be achieved undiminished with the devices described herein, and vice versa.

### BRIEF DESCRIPTION OF THE DRAWINGS

The present disclosure is exemplarily explained with regard to the accompanying drawings in which same reference numerals refer to the same or similar components. In the figures the following applies:

FIG. 1 shows in a simplified, schematic representation a blood treatment apparatus with an extracorporeal blood circuit in a first embodiment or the representation of a flow diagram of a blood treatment apparatus, exemplarily embodied as a hemodiafiltration apparatus;

FIG. 2 shows a schematic representation of an exemplary implementation of the first phase of a method; and

FIG. 3 shows a schematic representation of an exemplary implementation of the second phase of the method of FIG. 2.

### DETAILED DESCRIPTION

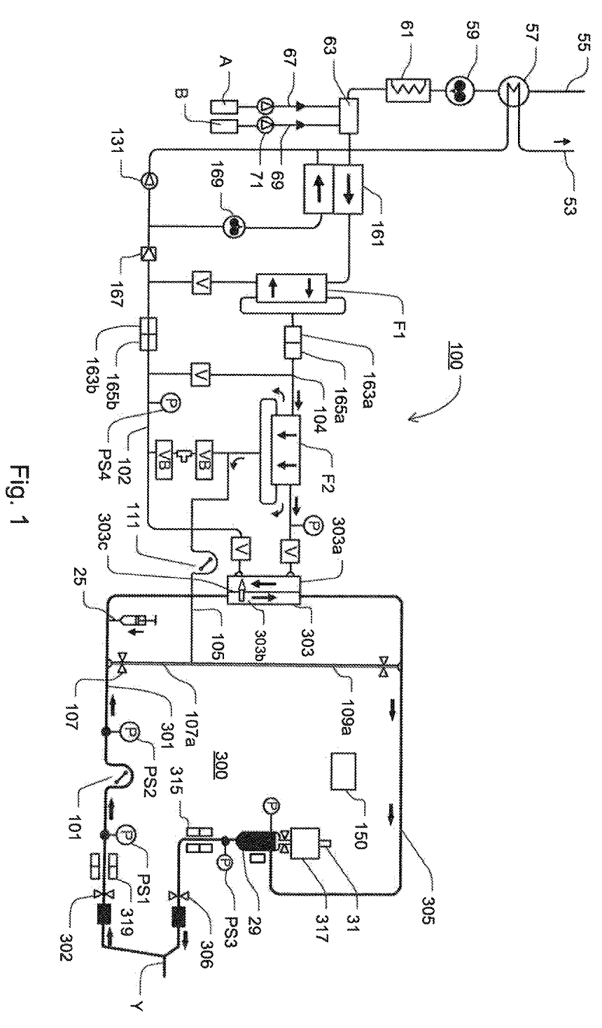

FIG. 1 shows an extracorporeal blood circuit 300, which can be connected to the vascular system of the patient (not shown) for a treatment via double-needle access, or via single-needle access, using, for example, an additional Y-connector (reference numeral Y) as shown in FIG. 1. The blood circuit 300 can optionally be present in sections thereof in or on a blood cassette.

Pumps, actuators and/or valves in the area of the blood circuit 300 are connected to a blood treatment apparatus 100 or for example, to a control device 150 comprised within.

The blood circuit 300 comprises (or is connected to) an arterial patient tubing clamp 302 and an arterial connection needle of an arterial section or of an arterial patient line, a blood withdrawal line or a first line 301. The blood circuit 300 further comprises (or is connected to) a venous patient tubing clamp 306 and a venous connection needle of a venous section, a venous patient line, blood return line or second line 305.

A blood pump 101 is provided in or on the first line 301, a substituate pump 111 is connected to a dialysis liquid inlet line 104 for conveying fresh dialysis liquid, which is filtered through a further filter (F2) (substituate). A substituate line 105 can be in fluid communication with the inlet line 104. Using the substituate pump 111, substituate can be introduced by pre-dilution via a pre-dilution valve 107, or by post-dilution via a post-dilution valve 109, into line sections via corresponding lines 107a or 109a, for example into the arterial line section 301 or into the venous line section 305 (here between a blood chamber 303b of a blood filter 303 and a venous air separation chamber or venous blood chamber 29) of the blood circuit 300

The blood filter 303 comprises the blood chamber 303b which is connected to the arterial line section 301 and the venous line section 305. A dialysis liquid chamber 303a of the blood filter 303 is connected to the dialysis liquid inlet line 104 leading to the dialysis liquid chamber 303a and a dialysate outlet line 102 leading away from the dialysis liquid chamber 303a which guides dialysate, i.e., used dialysis liquid. Dialysis liquid chamber 303a and blood chamber 303b are separated by a mostly semi-permeable membrane 303c. This membrane is what separates the blood side with the extracorporeal blood circuit 300 and the machine side with the dialysis liquid circuit or dialysate circuit, which is shown in FIG. 1 to the left of the membrane 303c.

The arrangement in FIG. 1 encompasses an optional detector 315 for detecting air and/or blood. The arrangement in FIG. 1 further encompasses one or two pressure sensors PS1 (upstream of the blood pump 101) and PS2 (downstream of the blood pump 101, which measures the pressure upstream of the blood filter 303 (“pre-hemofilter”)) at the positions shown in FIG. 1. Further pressure sensors may be provided, e.g., the pressure sensor PS3 downstream of the venous blood chamber 29.

An optional single-needle chamber 317 is used in FIG. 1 as a buffer and/or a compensating reservoir in a single-needle procedure in which the patient is connected to the extracorporeal blood circuit 300 via only one of the two blood lines 301, 305.

The arrangement in FIG. 1 additionally comprises an optional detector 319 for detecting air bubbles and/or blood.

An addition point 25 for Heparin may be optionally provided. Shown on the left in FIG. 1 is a mixing apparatus 63 being a dialysis liquid preparation system which, from containers A (for A concentrate via the concentrate supply 67) and B (for B concentrate via the concentrate supply 69), provides a predetermined mixture for the respective solution for use by the blood treatment apparatus 100 provides.

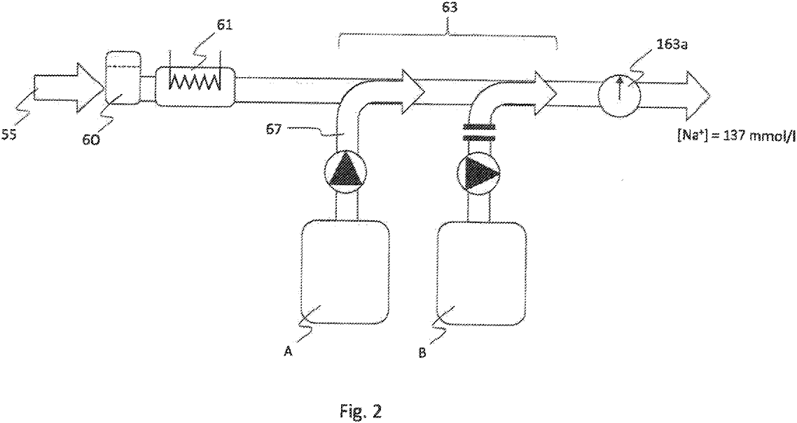

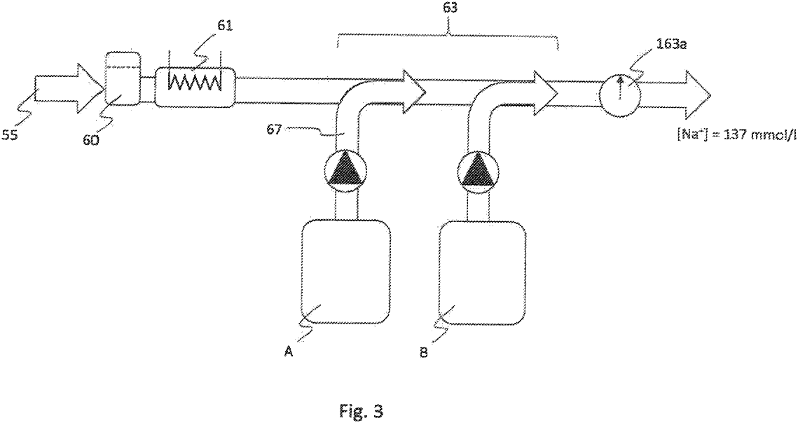

Alternatively, the mixing apparatus 63 as such can be omitted, and the two concentrates can be delivered successively into any fluid line and may come into contact with each other in any component of the blood treatment apparatus 100. After having been brought together in a common vessel (line, tubing, chamber, and so on), the so generated solution may be optionally thoroughly mixed or stirred, e.g., in a device 161 for balancing (e.g., a balancing chamber) as set forth below. Hence, the mixing apparatus 63 as mentioned herein is not restricted to a chamber as shown in FIG. 1. Rather, it may be any other vessel such as the fluid line denoted with 63 in FIG. 2 or FIG. 3

The solution may contain, in the heater 61 for example, warmed water from the water source 55 (on-line, e.g., as reverse osmosis water or from bags).

A pump 71, that may be referred to as a concentrate pump or sodium pump is in fluid communication with the mixing apparatus 63 and with a source having sodium, such as the container B, and/or conveys therefrom.

Furthermore, an outlet 53 for the effluent can be seen in FIG. 1. An optional heat exchanger 57 and a first flow pump 59, which is suitable for de-gassing, may complete the arrangement shown.

A further pressure sensor for measuring the filtrate pressure or the membrane pressure of the blood filter 303 may be provided as PS4 downstream of the blood filter 303 on the water-side, however preferably upstream of the ultrafiltration pump 131 in the dialysate outlet line 102. Further optional pressure measuring points P may also be provided.

Blood, which leaves the blood filter 303, passes through an optional venous blood chamber 29, which can comprise a de-aeration device 31 and/or may be in fluid communication with a further pressure sensor PS3.

The exemplary arrangement shown in FIG. 1 comprises a control device or closed-loop control device 150. This can be in wired or wireless signal communication with any of the components referred to herein - in particular or especially to the blood pump 101 - in order to control or regulate the blood treatment apparatus 100. It is optionally configured in order to carry out the method described herein, particularly automatically.

By using the device for on-line mixing of the dialysis liquid, a variation in the sodium content thereof controlled by the control device 150, is possible within certain limits. For this purpose, measurements determined via the conductivity sensors 163a, 163b may particularly be taken into account. Should an adjustment of the sodium content of the dialysis liquid (sodium concentration) or of the substituate be required or desired, this can be done by adjusting the delivery rate of the sodium pump 71.

Furthermore, the blood treatment apparatus 100 comprises means for conveying fresh dialysis liquid as well as dialysate. For this purpose, the first flow pump 59, which conveys fresh dialysis liquid towards the blood filter 303, is provided upstream of the blood filter 303. A first valve may be provided between the first flow pump 59 and the blood filter 303, which opens or closes the inlet to the blood filter 303 on the inlet side. A second optional flow pump 169 is provided, for example downstream of the blood filter 303, which conveys dialysate to the outlet 53. A second valve may be provided between the blood filter 303 and the second flow pump 169, which opens or closes the outlet on the outlet side.

Furthermore, the blood treatment apparatus 100 optionally comprises a device 161 for balancing the flow going into and coming out of the dialyzer 303 on the machine side. The device 161 for balancing is preferably arranged in a line section between the first flow pump 59 and the second flow pump 169.

The blood treatment apparatus 100 further encompasses means, such as the ultrafiltration pump 131, for the precise removal of a fluid volume from the balanced circuit as specified by the user and/or by the control device 150.

Sensors such as the optional conductivity sensors 163a, 163b serve to determine the conductivity, which in some embodiments is temperature-compensated, as well as the liquid flow upstream and downstream of the dialyzer 303.

Temperature sensors 165a, 165b can be provided individually or in groups. Temperature readings supplied by them can be used to determine a temperature-compensated conductivity.

A leakage sensor 167 is optionally provided.

Further flow pumps, in addition or as an alternative to, the one indicated with the reference numeral 169 for example, can also be provided.

A row of optional valves is each indicated in FIG. 1 with a V. By pass valves are indicated with a VB.

In some embodiments the control device 150 determines the electrolyte and/or liquid balancing based on the measurement readings of the afore-mentioned, optional sensors.

Filters F1 and F2 may be provided in series-connection.

The filter F1 here serves exemplarily, via the mixing apparatus 63, to produce sufficiently pure dialysis liquid, even when using impure water, which then flows through the blood filter 303, e.g., according to the counter-current principle.

Exemplarily, here the filter F2 serves to generate a sterile or sufficiently filtered substituate from the sufficiently pure dialysis liquid, which leaves the first filter F1, by filtering out pyrogenic substances, for example. This substituate can safely be added to the patient’s blood flowing extracorporeally and thus ultimately be supplied to the patient’s body.

The blood treatment apparatus 100 described herein is shown in FIG. 1 as a device for hemo(dia)filtration. However, hemodialysis devices also fall within the scope of the present disclosure, even though they are not specifically shown in the figures.

The present disclosure is not limited to the embodiment described above, this serves only as an illustration.

The arrows shown in FIG. 1 generally indicate the direction of flow in FIG. 1.

FIG. 2 shows a schematic representation of an exemplary implementation of the first phase of the method described herein. The components shown are all part of the hydraulic unit of the blood treatment apparatus 100. In particular, FIG. 1 shows the mixing apparatus 63 used for preparing the priming liquid. As stated before, the mixing apparatus 63 is not limited to the one shown in FIG. 1. Rather, the mixing can take place anywhere within the blood treatment apparatus 100 or the tubing attached to the blood treatment apparatus 100. In the example of FIG. 2, instead of feeding concentrates from sources A and B into a mixing apparatus 63 such as the one shown in FIG. 1, a fluid line into which the concentrates may be fed downstream the water source 55 is used as a mixing chamber.

During the first phase, the priming liquid contains no bicarbonate, a priming takes place with acid concentrate diluted in water only.

Water gained from a reverse-osmosis (RO) process or any other type of water such as tap water enters the mixing apparatus 63 from the water source 55.

Having passed an optional degassing device 60 the water may be heated in a heating apparatus 61 such that the temperature of the eventually generated priming liquid is between 25° C. and 35° C. (degrees Celsius).

Downstream of the heating apparatus 61 acid concentrate from source A is pumped into the water flow in the mixing apparatus 63.

As indicated by the symbol denoting the pump corresponding the source B of bicarbonate concentrate and the representation of the valve right downstream of the pump no bicarbonate is mixed into the thus generated water/acid solution.

A conductivity cell 163a may be used to monitor and control the [Na⁺] concentration of the priming liquid prepared in the mixing apparatus 63. In the first phase, a conductivity of 14.0 [mS] is measured.

A desirable [Na⁺] concentration is 137 mmol/l. As can be seen from FIG. 2, the [Na⁺] concentration of the priming liquid originates exclusively from the source A since no fluid and, hence, no sodium chloride or sodium [Na⁺] is contributed to the priming liquid from source B.

In the particular embodiment of FIG. 2, the container A comprises an acid concentrate for dialysis with sodium chloride in a concentration of 263 g/l NaCl. Further the container A comprises an acid, like citric acid or acetic acid, and preferably one or more of the following electrolytes, like magnesium chloride, calcium chloride and potassium chloride. For priming the extracorporeal circuit, the concentrate is diluted with water (33:1) to result in a priming solution comprising 137 mmol/l sodium.

FIG. 3 shows a schematic representation of an exemplary implementation of the second phase of the method of FIG. 2. The components shown in FIG. 3 are those of FIG. 2.

In the second phase bicarbonate concentrate is added to the priming liquid in preparation of the extracorporeal blood circuit 300 as is indicated by the icon representing the pump corresponding to the source B. In the particular setting shown in FIG. 3, the container A comprises 263 g/l NaCl. Its concentrate is used with a dilution factor 45:1 contributing 100 mmol/l sodium chloride, whereas the flow from container B contributes 37 mmol/l in an exemplary dilution having a dilution factor 25:1.

As in the first phase, the [Na⁺] concentration of the priming liquid is 137 mmol/l. However, the contribution from container A amounts to 100 mmol/l [Na⁺] and is, hence, less than before. In contrast to the first step, sodium chloride is contributed from the container B as well. In the example of FIG. 3, it also provides the lacking 37 mmol/l [Na⁺] needed for preparing a priming solution having a concentration of 137 mmol/l [Na⁺] as in the first phase.

The bicarbonate concentrate of container B may be saturated. In a saturated condition of the bicarbonate concentration of container B its sodium bicarbonate concentration may, in one exemplary embodiment, amount to 95.5 g/l at 20°. Those number are, however, not intended to be limiting, of course.

It is noted that the priming solution prepared in the first phase, which is exemplary described with respect to FIG. 2, may be used when circulating a liquid through parts of the extracorporeal blood circuit 300 and/or of the hydraulic system while a patient is temporarily disconnected from the blood treatment apparatus 100. Also, a solution used for rinsing parts of the extracorporeal blood circuit 300 and/or of the hydraulic system between two patients that are consecutively treated with more or less the same equipment. Before connecting the next patient to said equipment, the solution prepared by the mixing apparatus 63 will have been mixed as set forth with respect to the second phase, e.g., as described supra.

List of reference numerals

| | |
|---|---|
| 25 | addition point for Heparin (optional) |
| 29 | venous blood chamber (optional) |
| 31 | de-aeration device |
| 53 | outlet |
| 55 | water source |
| 57 | heat exchanger |
| 59 | first flow pump |
| 60 | degassing device |
| 61 | heating apparatus |
| 63 | mixing apparatus |
| 67 | concentrate supply |
| 69 | concentrate supply |
| 71 | concentrate pump; sodium pump |
| 100 | blood treatment apparatus |
| 101 | blood pump |
| 102 | dialysate outlet line, effluent inlet line |
| 104 | dialysis liquid inlet line |
| 105 | substituate line |
| 107 | pre-dilution valve |
| 107 a | line |
| 109 | post-dilution valve |
| 109 a | line |
| 111 | pump for substituate |
| 121 | pump for dialysis liquid |
| 131 | pump for dialysate or effluent |
| 150 | control device or closed-loop control device |
| 161 | device |
| 163 a | conductivity sensor |
| 163 b | conductivity sensor |
| 165 a | temperature sensor |
| 165 b | temperature sensor |
| 167 | leakage sensor |
| 169 | second flow pump |
| 300 | extracorporeal blood circuit |
| 301 | first line (arterial line section) |
| 302 | first tubing clamp |
| 303 | blood filter or dialyzer |
| 303 a | dialysis liquid chamber |
| 303 b | blood chamber |
| 303 c | semi-permeable membrane |
| 305 | second line (venous line section) |
| 306 | (second) tubing clamp |
| 315 | detector |
| 317 | single-needle chamber |
| 319 | detector |
| F1 | filter |
| F2 | filter |
| [Na⁺] | sodium concentration |
| A | container (source of acid) |
| B | container (source of bicarbonate) |
| P | pressure measuring points |
| PS1 | arterial pressure sensor (optional) |
| PS2 | arterial pressure sensor (optional) |
| PS3 | pressure sensor (optional) |
| PS4 | pressure sensor for measuring the filtrate pressure (optional) |
| V | valves |
| VB | bypass valves |
| Y | Y-connector |

1-12. (canceled)

13. A method of priming an extracorporeal blood circuit using a blood treatment apparatus comprising a dialysis liquid preparation system having a source of water, a source of bicarbonate concentrate and a source of acid concentrate including sodium chloride while not having or being connected to a source that comprises sodium chloride only or exclusively, the blood treatment apparatus configured to prepare a dialysis liquid from water, the bicarbonate concentrate and the acid concentrate only, wherein the extracorporeal blood circuit comprises an arterial line section, connectable to a patient, for drawing blood from the patient, a venous line section, connectable to the patient, for returning the blood to the patient, and a blood side compartment or blood chamber of a blood filter, the method comprising a first phase comprising:
preparing a priming solution from the source of water and the source of acid concentrate only to obtain an acid/water solution, wherein bicarbonate from the source of bicarbonate concentrate is absent from the solution,
connecting the arterial line section to an outlet of the dialysis liquid preparation system of the blood treatment apparatus and,
filling the extracorporeal blood circuit with the priming solution.

14. The method according to claim 13, wherein during the first phase the temperature of the priming solution is kept below 35° C.

15. The method according to claim 13, wherein during the first phase the temperature of the priming solution is kept between 25° C. and 30° C.

16. The method according to claim 15, wherein the method further comprises a second phase, following the first phase, the second phase comprising:
adding bicarbonate from the source of bicarbonate concentrate to the priming solution.

17. The method according to claim 16, wherein during the second phase the temperature of the priming solution is raised to at least 35° C.

18. The method according to claim 16, wherein the priming is continued for at least 5 minutes before the priming is considered as completed.

19. The method according to claim 18, wherein circulation of the priming solution within the extracorporeal circuit is continued for at least 5 minutes before the priming is considered as completed.

20. A control device or closed-loop control device configured to carry out or to initiate the method according to claim 13 in interaction with the blood treatment apparatus.

21. A blood treatment apparatus for the extracorporeal treatment of a patient’s blood, comprising the control device or closed-loop control device according to claim 20.

22. The blood treatment apparatus according to claim 21, wherein the blood treatment apparatus comprises a hemodialysis apparatus, hemofiltration apparatus or hemodiafiltration apparatus.

23. The blood treatment apparatus according to claim 21, wherein the blood treatment apparatus is configured for acute or chronic renal replacement therapy.

24. The blood treatment apparatus according to claim 23, further comprising sensors arranged and configured to measure a sodium concentration, in the priming solution.
